(19) United States
(12) Patent Application Publication
(10) Pub. No.: US 2026/0026838 A1
(43) Pub. Date: Jan. 29, 2026

(54) METHOD AND SYSTEM FOR DETERMINING A POSITION OF A NEEDLE TIP

(71) Applicant: B. Braun Melsungen AG, Melsungen (DE)

(72) Inventor: Jens Wildhagen, Hannover (DE)

(21) Appl. No.: 19/276,439

(22) Filed: Jul. 22, 2025

(30) Foreign Application Priority Data
Jul. 24, 2024 (DE) 10 2024 120 960.7

(51) Int. Cl. A61B 17/34

(52) U.S. Cl. CPC A61B 17/3403; A61B 2017/3413

(57) ABSTRACT

A method for determining a position of a needle tip inside a body includes: providing an ultrasound head of an imaging system, providing a transmission device having multiple position transmitters, and securing the transmission device to the ultrasound head in a defined position. Multiple ultrasound position signals are coupled into the body by the position transmitters. The signals are received by a sensor attached to a needle located in the body and arranged at a known axial distance from the needle tip. Signal transit times are determined by an evaluation device. The position of the needle tip relative to the ultrasound head is determined by the evaluation device depending on the signal transit times, the position of the transmission device relative to the ultrasound head, and the axial distance between the sensor and the needle tip. The method can be performed with a medical system.

### CROSS-REFERENCE TO RELATED APPLICATION

This application claims priority under 35 U.S.C. § 119 to German Application No. 10 2024 120 960.7, filed on Jul. 24, 2024, the content of which is incorporated by reference herein in its entirety.

### FIELD

The present disclosure relates to a method and a system for determining a position of a needle tip inside a body.

### BACKGROUND

Such methods and systems are used, inter alia, in the field of regional anaesthesia. From the prior art, a system marketed under the registered trademark ONVISION® is configured to display the position of the needle tip in an image plane of an ultrasound image. The known system is fully integrated into an underlying imaging ultrasound system.

### SUMMARY

It is an object of the present disclosure to provide a method and a system of the type mentioned at the beginning and which offer advantages over the prior art.

The method according to the present disclosure comprises the steps: providing an ultrasound head of an imaging ultrasound system; providing a transmission device having multiple ultrasound position transmitters and at least one securing portion; securing the transmission device to the ultrasound head, wherein the transmission device is secured to the ultrasound head releasably and in a defined relative position to the ultrasound head by means of the securing portion; coupling multiple ultrasound position signals into the body, wherein the ultrasound position signals are coupled in by means of the multiple ultrasound position transmitters; receiving the ultrasound position signals, wherein the ultrasound position signals are received by means of a sensor attached to a needle located in the body and arranged at a known axial distance from the needle tip thereof; determining signal transit times of the ultrasound position signals, wherein the signal transit times are determined by means of an evaluation device connected to the sensor and the transmission device; determining the position of the needle tip in relation to the ultrasound head, wherein the position is determined by means of the evaluation device depending on the signal transit times, the relative position of the transmission device in relation to the ultrasound head and the axial distance between the sensor and the needle tip.

The method according to the present disclosure does not need to be integrated into the imaging ultrasound system and is therefore particularly universally usable. Instead of a software-based and/or hardware-based integration into the imaging ultrasound system, the method according to the present disclosure provides the releasable securing of the transmission device to the ultrasound head of the imaging ultrasound system. For securing to the ultrasound head, the transmission device comprises the at least one securing portion, which can be secured releasably in a force-fitting and/or form-fitting manner to the ultrasound head. The securing is carried out in a defined relative position to the ultrasound head. The ultrasound position transmitters of the transmission device are configured to couple the multiple ultrasound position signals into the body. These ultrasound position signals are received by means of the sensor attached to the needle, wherein, in one embodiment of the present disclosure, multiple sensors are attached to the needle. The position of the needle tip is actually determined by means of the evaluation device. Said evaluation device is connected to the at least one sensor and the transmission device, for example via a wired or wireless connection. To determine the position of the needle tip, the signal transit times of the ultrasound position signals between the individual ultrasound position transmitters and the at least one sensor are first determined. A person skilled in the art is familiar with different approaches for determining signal transit times, and so further details on this need not be discussed in more detail.

If the signal transit times are known, it is possible to conclude a respective distance between the individual ultrasound position transmitters and the at least one sensor at the needle tip, in particular depending on a frequency and/or propagation speed of the ultrasound position signals. The position of the needle tip is then determined depending on the determined signal transit times and/or distances, the relative position of the transmission device in relation to the ultrasound head, and the axial distance between the at least one sensor and the needle tip. The at least one sensor is preferably arranged on the needle tip, such that the axial distance is practically zero or negligibly small in terms of the accuracy of the position determination. The position is determined on the basis of known geometric, in particular trigonometric, relationships. In embodiments with multiple sensors, these are arranged at known axial distances from the needle tip. In this case, an orientation of the needle can also be determined. The orientation can be determined on the basis of known geometric, in particular trigonometric, relationships by means of the evaluation device. In addition, the multiple sensors allow a more accurate position determination. Furthermore, the multiple sensors possibly also arranged further away from the needle tip prevent the function of needle tip from being impaired by the presence of a sensor placed as close as possible to the needle tip.

The method according to the present disclosure is particularly suitable for use in regional anaesthesia.

In one embodiment, at least one first ultrasound position signal is coupled in by a first ultrasound position transmitter and a second ultrasound position signal is coupled in by a second ultrasound position transmitter. In this embodiment, the transmission device thus comprises at least two ultrasound position transmitters, namely the first ultrasound position transmitter and the second ultrasound position transmitter. This embodiment thus enables the determination of the position with respect to two coordinate axes, i.e. in one plane. Such a planar position determination may already be sufficient for some use cases.

In a further embodiment, a third ultrasound position signal is coupled in by a third ultrasound position transmitter. In this embodiment, the transmission device thus comprises at least three ultrasound position transmitters, namely the first ultrasound position transmitter, the second ultrasound position transmitter and the third ultrasound position transmitter. This embodiment allows a position determination of the needle tip with respect to three coordinate axes. Such a spatial position determination has more significance than a planar position determination. In a further embodiment, the transmission device comprises more than three ultrasound position transmitters, for example four, five, six or more than six. In this case, a corresponding number of ultrasound position signals are coupled in. This can enable further improved accuracy of the position determination.

In a further embodiment, the ultrasound position signals have a frequency that differs from a transmission frequency of the ultrasound head. The frequency of the ultrasound position signals, which is different from the transmission frequency of the ultrasound head, prevents the imaging ultrasound method from being impaired by the position determination. Such an impairment may occur if the frequency of the ultrasound position signals and the transmission frequency of the ultrasound head are identical or very similar. In one embodiment, the ultrasound position signals are coupled in/transmitted at the same frequency. In a further embodiment, the ultrasound position signals have different frequencies. In a further embodiment the ultrasound position signals are coupled in in a manner synchronized in time with the ultrasound signal of the ultrasound head, for example with a time offset. In one embodiment, the ultrasound position signals and the ultrasound signal of the imaging ultrasound method are intermittently coupled in. For example, so-called frames can be identified on the basis of the received ultrasound signal of the imaging ultrasound method and the temporal transmission of the ultrasound position signals can be adapted and/or synchronized accordingly in order to avoid impairments in the imaging ultrasound method.

In a further embodiment, the securing portion is latched to a complementary securing portion of the ultrasound head. As an alternative or in addition, the securing portion is plugged to a/the complementary securing portion. In this embodiment, the transmission device is therefore secured to the ultrasound head with a releasable latching connection and/or a releasable plug connection. In one embodiment, the securing portion is a latching portion. In a further embodiment, the securing portion is alternatively or additionally a plug portion. The same applies mutatis mutandis to the complementary securing portion of the ultrasound head. It is understood that the transmission device may have multiple securing portions. The ultrasound head may also have multiple complementary securing portions. The complementary securing portion of the ultrasound head is preferably formed by the outer contour thereof. The at least one securing portion of the transmission device is matched in this case to the outer contour of the ultrasound head. This means that a constructive adjustment of the ultrasound head for the purpose of securing the transmission device can be omitted.

In a further embodiment, the method comprises a further step of: outputting the position, wherein the position is output acoustically and/or visually perceptibly by means of an output device. In one embodiment, the output device has a speaker configured for acoustic output of the position. In a further embodiment, the output device alternatively or additionally has a screen configured for the visually perceptible output of the position. For example, the position can be displayed graphically and/or in a text-based manner. In one embodiment, the output device is a component of the imaging ultrasound system, wherein the evaluation device is configured for connection to the output device of the imaging ultrasound system.

In a further embodiment, no ultrasound signals, in particular ultrasound position signals, are transmitted from the needle. Ultrasound signals or ultrasound position signals are only received, specifically by means of the at least one sensor attached to the needle. In other words: the needle is passive or a passive medical product. In contrast to a basically conceivable and possible coupling of ultrasound signals from the needle, there are advantages, in particular improved patient safety.

The medical system according to the present disclosure is configured to determine a position of a needle tip inside a body and has a transmission device, a needle and an evaluation device. The medical system is preferably configured to carry out the method according to the present disclosure and/or the embodiments thereof. The transmission device has multiple ultrasound position transmitters and at least one securing portion. The securing portion is configured for releasable securing to a complementary securing portion of an ultrasound head. The ultrasound head is a component of an imaging ultrasound system. The ultrasound head is not a component of the medical system. The ultrasound position transmitters are each configured to couple an ultrasound position signal into the body. The needle comprises the needle tip and at least one sensor. The sensor is configured to receive the ultrasound position signals. The sensor is arranged at a known axial distance from the needle tip. The axial distance is preferably zero or at least negligibly small in terms of the position determination. In other words: the sensor is preferably arranged directly on the needle tip. The evaluation device is connected to the sensor and the transmission device. The connection may be wired and/or wireless. The evaluation device is configured to determine the signal transit times of the ultrasound position signals. The evaluation device is also configured to determine the position of the needle tip in relation to the ultrasound head, wherein the determination is carried out depending on the signal transit times, a defined relative position of the transmission device to the ultrasound head, and the axial distance of the sensor from the needle tip.

Further features and advantages of the medical system result from the disclosure of the method according to the present disclosure and vice versa. The same applies to the respective embodiments of the medical system according to the present disclosure and the method according to the present disclosure.

In a further embodiment, the at least one securing portion is a latching portion configured for latching to an outer contour of the ultrasound head. As an alternative or in addition, the at least one securing portion is a plug portion configured for plugging to a/the outer contour of the ultrasound head. In this embodiment of the present disclosure, the outer contour of the ultrasound head forms the complementary securing portion for securing the transmission device. A constructive adaptation of the ultrasound head to the medical system, in particular to the transmission device thereof, can therefore be omitted. This allows a particularly universal use of the medical system.

In a further embodiment, the transmission device comprises at least two ultrasound position transmitters. This allows a planar position determination with respect to two coordinate axes.

In a further embodiment, the transmission device comprises at least three ultrasound position transmitters. This enables a spatial position determination with respect to three coordinate axes.

In a further embodiment, the needle does not have a transmission device for transmitting ultrasound signals, specifically ultrasound position signals. The needle is therefore configured exclusively for receiving ultrasound signals, more specifically the ultrasound position signals, and for this purpose comprises the at least one sensor. The needle is therefore passive or a passive medical product. In contrast to a basically conceivable and possible coupling of ultrasound signals from the needle, there are advantages, in particular improved patient safety.

### BRIEF DESCRIPTION OF THE DRAWINGS

Further advantages and features of the present disclosure follow from the following description of preferred exemplary embodiments, which are shown in the drawings.

FIG. 1 shows a schematic block illustration of an embodiment of a method according to the present disclosure for determining a position of a needle tip inside a body,

FIG. 2 shows a schematic illustration of an embodiment of a medical system according to the present disclosure which is configured to carry out the method according to FIG. 1,

FIG. 3 shows a schematically simplified illustration of an exemplary use situation of the medical system according to FIG. 2 and

FIG. 4 shows an illustration, corresponding to FIG. 3, of a further exemplary use situation.

### DETAILED DESCRIPTION

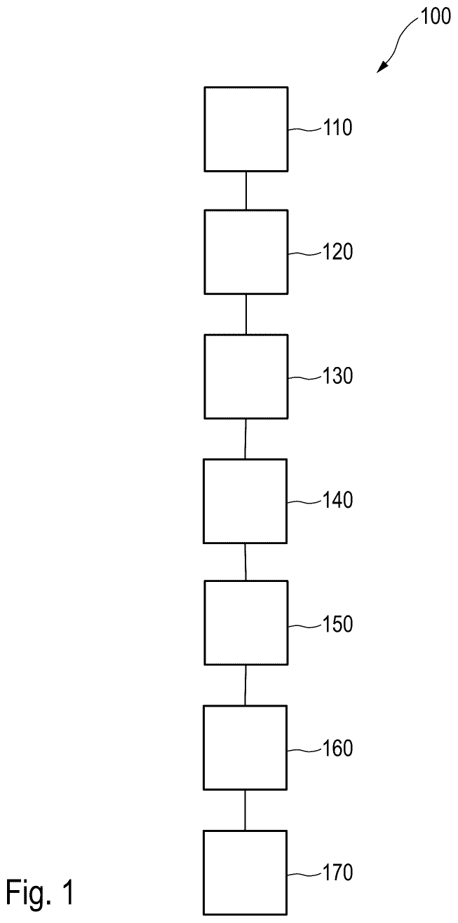

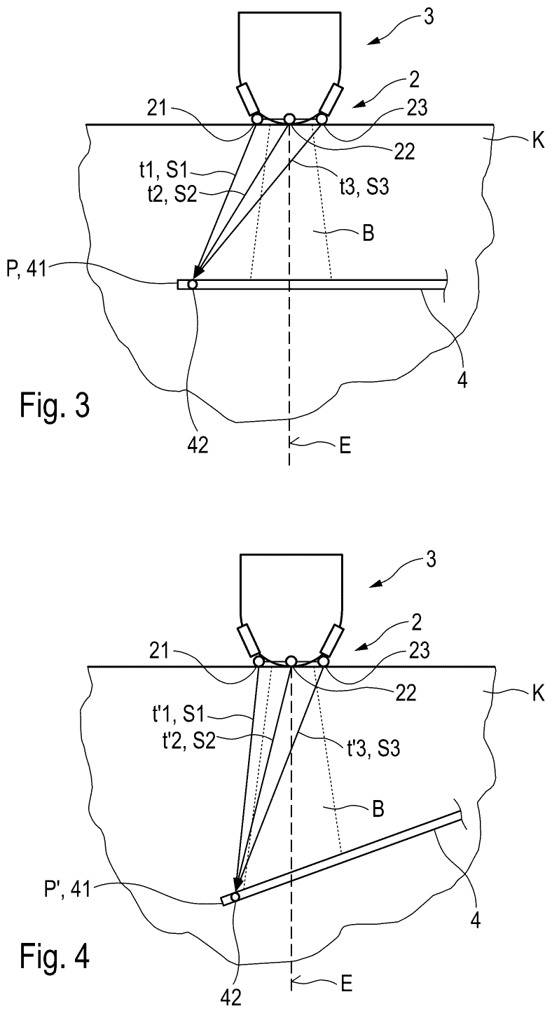

According to FIG. 1, a method 100 for determining a position P of a needle tip 41 inside a body K is provided (see FIG. 3, 4).

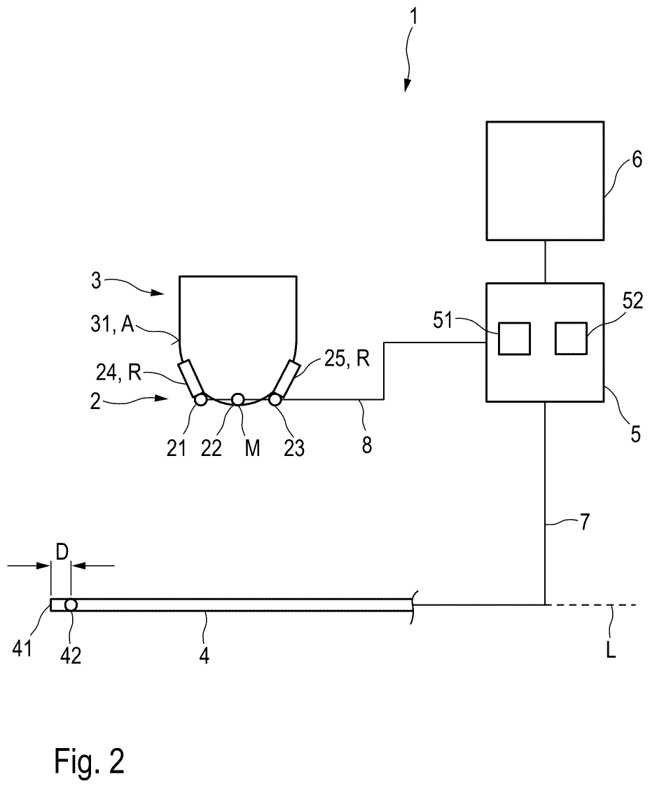

The method 100 can be carried out using the medical system 1 according to FIG. 2 and comprises steps 110 to 170.

The medical system 1 comprises a transmission device 2, a needle 4 and an evaluation device 5. In the embodiment shown, the medical system 1 also comprises an optional output device 6.

The transmission device 2 comprises multiple ultrasound position transmitters 21, 22, 23. The multiple ultrasound position transmitters 21, 22, 23 can also be referred to as the first ultrasound position transmitter 21, second ultrasound position transmitter 22 and third ultrasound position transmitter 23. The transmission device 2 also comprises at least one securing portion 24, 25. The ultrasound position transmitters 21, 22, 23 are each configured to transmit an ultrasound position signal S1, S2, S3. The ultrasound position signals S1, S2, S3 can also be referred to as the first ultrasound position signal S1, second ultrasound position signal S2 and third ultrasound position signal S3. The at least one securing portion 24, 25 is configured for releasable securing to a complementary securing portion 31 of an ultrasound head 3. The ultrasound head 3 is not part of the medical system 1, but rather a component of an imaging ultrasound system, as it is generally known in the field of medical technology.

The needle 4 comprises the needle tip 41 and at least one sensor 42. The needle tip 41 is distally arranged in a conventional manner. The sensor 42 is arranged at a known axial distance D from the needle tip 41. The axial distance D is small compared to an overall length of the needle 4. The axial distance D is extended along a longitudinal axis L of the needle 4. In one embodiment, not shown in the figures, multiple sensors are attached to the needle and each positioned at a known axial distance from the needle tip.

The evaluation device 5 is connected to the sensor 42 and the transmission device 2. A signal connection 7, which in the present case is a wired connection, is provided for connection to the sensor 42. A further signal connection 8, which is also wired, is provided for connection to the transmission device 2. As an alternative, wireless connections may be provided.

The method 100 first provides in step 110 for the provision of the ultrasound head 3.

In step 120, the transmission device 2 is provided.

In step 130, the transmission device 2 is secured to the ultrasound head 3. In this case, the transmission device 2 is releasably secured to the ultrasound head 3 by means of the at least one securing portion 24, 25. The securing is carried out here at the complementary securing portion 31.

The transmission device 2 is secured to the ultrasound head 3 in a defined relative position in relation to the ultrasound head 3. As a result, a position of the ultrasound position transmitters 21, 22, 23 in relation to the ultrasound head 3 is known. Said positions of the ultrasound position transmitters 21, 22, 23 can be defined, for example, in relation to a centre point M of the ultrasound head 3, which can also be referred to as the centre.

In the embodiment shown, the transmission device 2 comprises a first securing portion 24 and a second securing portion 25. The number in this case is to be understood as purely exemplary.

The two securing portions 24, 25 are each designed as a latching portion R in the embodiment shown. The latching portions R can be latched directly to an outer contour A of the ultrasound head 3. Accordingly, the outer contour A of the ultrasound head 3 in the present case forms said complementary securing portion 31 for releasable securing using the securing portions 24, 25 of the transmission device 2.

In an embodiment not shown in the figures, the securing portions are each designed as a plug portion.

The method 100 provides in step 140 a coupling of the ultrasound signals S1, S2, S3 into the body K.

In the exemplary use situations shown (see FIG. 3, 4), the ultrasound head 3, together with the transmission device 2 attached thereto, rests on the body K. For the actual imaging ultrasound method, in the present case, an ultrasound signal B is coupled into the body K by means of the ultrasound head 2, which ultrasound signal, in the present case, can also be referred to as a beam. The term “beam” is familiar to a person skilled in the art.

The beam B is coupled into the body K along an imaging plane E of the imaging ultrasound method. The image plane E extends in the present case along a depth direction and a longitudinal direction.

In contrast to the ultrasound signal B of the ultrasound head 3, the ultrasound position signals S1, S2, S3 of the ultrasound position transmitters 21, 22, 23 are not used for determining an ultrasound image, but for determining the position of the needle tip 41.

In step 150, the method 100 provides for receiving the ultrasound position signals S1, S2, S3. The ultrasound position signals S1, S2, S3 are received by means of the sensor 41.

The method 100 provides in step 160 a determination of signal transit times t1, t2, t3 of the ultrasound position signals S1, S2, S3. The signal transit times t1, t2, t3 are graphically symbolized in FIG. 3 with an arrow each and can also be referred to as the first signal transit time t1, second signal transit time t2 and third signal transit time t3. The first signal transit time t1 is the transit time of the first ultrasound position signal S1, i.e. the time that elapses between the transmission of the ultrasound position signal S1 by means of the first ultrasound position transmitter 21 and its reception by means of the sensor 42. The same applies mutatis mutandis for the second signal transit time t2 (transit time of the second ultrasound position signal S2) and the third signal transit time t3 (transit time of the third ultrasound position signal S3). The signal transit times t1, t2, t3 are determined by means of the evaluation device 5. For this purpose, the ultrasound position signals S1, S2, S3 are evaluated in a manner known to a person skilled in the art.

The method 100 provides in step 170 a determination of the position P of the needle tip 41 in relation to the ultrasound head 3, in particular in relation to the centre point M of the ultrasound head 3. The position P is determined here by means of the evaluation device 5 depending on the signal transit times t1, t2, t3. The signal transit times t1, t2, t3 allow a conclusion to be drawn regarding the respective distances between the ultrasound position transmitters 21, 22, 23 and the sensor 42 when the propagation speed of the ultrasound position signals S1, S2, S3 is known. The position P is then determined depending on the signal transit times t1, t2, t3 and on the basis of simple geometric, in particular trigonometric, relationships.

FIG. 4 shows a further use situation in which the needle 4 occupies a different position in relation to the ultrasound head 3. Accordingly, the needle tip 41 occupies a modified position P′. In addition, there are modified signal transit times t′1, t′2, t′3.

In this case, the position P (or the modified position P′) can be determined spatially and with respect to three coordinate axes in relation to the centre point M. If only two ultrasound position transmitters are available, the position can be determined with respect to two coordinate axes, and this can then also be referred to as a planar position determination.

In the embodiment shown, the ultrasound position signals S1, S2, S3 are transmitted at a frequency that differs from a transmission frequency of the ultrasound head 3 and thus from the frequency of the ultrasound signal B. This prevents the ultrasound position signals S1, S2, S3 from interfering with the ultrasound imaging method.

In the embodiment shown, the method also provides for an outputting of the position P by means of the output device 6. The output device 6 is configured for the acoustic and/or visually perceptible output of the position P. For this purpose, the output device 6 may comprise, for example, a speaker (acoustic output) or a screen (visual output). In one embodiment, the output device 6 is assigned to the imaging ultrasound system which also comprises the ultrasound head 3. In this case, the evaluation device 5 can be connected to the output device 6.

In the present case, the evaluation device 5 also comprises a memory unit 51 and a processor unit 52. The processor unit 52 is configured at least to carry out steps 160, 170.

1. A method for determining a position of a needle tip of a needle inside a body, the method comprising the steps of:
providing an ultrasound head of an imaging ultrasound system;
providing a transmission device comprising multiple ultrasound position transmitters and at least one securing portion;
securing the transmission device to the ultrasound head, wherein the transmission device is secured to the ultrasound head releasably and in a defined position relative to the ultrasound head by the at least one securing portion;
coupling multiple ultrasound position signals into the body, wherein the multiple ultrasound position signals are coupled in by the multiple ultrasound position transmitters;
receiving the multiple ultrasound position signals, wherein the multiple ultrasound position signals are received by at least one sensor attached to the needle and arranged at a known axial distance from the needle tip;
determining signal transit times of the multiple ultrasound position signals, wherein the signal transit times are determined by an evaluation device connected to the at least one sensor and the transmission device; and
determining the position of the needle tip in relation to the ultrasound head, wherein the position of the needle tip is determined by the evaluation device depending on the signal transit times, the position of the transmission device relative to the ultrasound head, and the known axial distance between the at least one sensor and the needle tip.

2. The method according to claim 1, wherein:
at least one first ultrasound position signal of the multiple ultrasound position signals is coupled in by a first ultrasound position transmitter of the multiple ultrasound position transmitters, and
a second ultrasound position signal of the multiple ultrasound position signals is coupled in by a second ultrasound position transmitter of the multiple ultrasound position transmitters.

3. The method according to claim 2, wherein a third ultrasound position signal of the multiple ultrasound position signals is coupled in by a third ultrasound position transmitter of the multiple ultrasound position transmitters.

4. The method according to claim 1, wherein the multiple ultrasound position signals have a frequency that differs from a transmission frequency of the ultrasound head.

5. The method according to claim 1, wherein the at least one securing portion is latched and/or plugged to a complementary securing portion of the ultrasound head.

6. The method according to claim 1, further comprising the step of outputting the position of the needle tip, wherein the position of the needle tip is output acoustically and/or visually perceptibly by an output device.

7. The method according to claim 1, wherein no ultrasound signals are transmitted from the needle.

8. A medical system comprising:
a needle comprising a needle tip for insertion into a body and at least one sensor;
a transmission device; and
an evaluation device,
the transmission device having multiple ultrasound position transmitters and at least one securing portion,
the at least one securing portion configured for releasably securing to a complementary securing portion of an ultrasound head of an imaging ultrasound system,
the multiple ultrasound position transmitters each configured for coupling an ultrasound position signal into the body,
the at least one sensor configured to receive the multiple ultrasound position signals and arranged at a known axial distance from the needle tip,
the evaluation device being connected to the at least one sensor and the transmission device,
the evaluation device configured to determine signal transit times of the multiple ultrasound position signals;
the evaluation device further configured to determine a position of the needle tip relative to the ultrasound head based on the signal transit times, a defined position of the transmission device relative to the ultrasound head, and the known axial distance.

9. The medical system according to claim 8, wherein the at least one securing portion is a latching portion configured to latch with an outer contour of the ultrasound head.

10. The medical system according to claim 8, wherein the at least one securing portion is a plug portion configured for plugging to an outer contour of the ultrasound head.

11. The medical system according to claim 8, wherein the transmission device comprises at least two ultrasound position transmitters.

12. The medical system according to claim 8, wherein the transmission device comprises at least three ultrasound position transmitters.

13. The medical system according to claim 8, wherein the needle does not have any transmission devices for transmitting ultrasound signals.
